(19) United States
(12) Patent Application Publication
(10) Pub. No.: US 2014/0370563 A1
(43) Pub. Date: Dec. 18, 2014

(54) COMPOSITIONS AND METHODS FOR THE RELIEF OF INHIBITION OF ALDEHYDE DECARBONYLASE

(71) Applicants: John Shanklin, Shoreham, NY (US); Carl Andre, Raleigh, NC (US)

(72) Inventors: John Shanklin, Shoreham, NY (US); Carl Andre, Raleigh, NC (US)

(73) Assignee: BROOKHAVEN SCIENCE ASSOCIATES, LLC, Upton, NY (US)

(21) Appl. No.: 14/240,836

(22) PCT Filed: Aug. 24, 2012
(86) PCT No.: PCT/US12/52222

§ 371 Date: Sep. 4, 2014

Related U.S. Application Data
(60) Provisional application No. 61/527,630, filed on Aug. 26, 2011.

(51) Int. Cl. C12P 5/02

(52) U.S. Cl. CPC C12P 5/026; C12P 5/02
USPC 435/167; 435/192; 435/252.33; 435/320.1; 435/257.2

(57) ABSTRACT

The present invention is related to compositions and methods for enhanced synthesis of hydrocarbons, particularly, but not limited to, alkanes. The invention, in one embodiment, utilizes the co-expression of a hydrogen peroxide metabolizing enzyme in the presence of an aldehyde decarbonylase enzyme to relieve hydrogen peroxide inhibition of the aldehyde decarbonylase enzyme by hydrogen peroxide. In a preferred embodiment a catalase-aldehyde decarbonylase expression construct and fusion peptide is used. The present invention also relates to microorganisms engineered to express said enzymes and to produce hydrocarbon molecules.

This application claims the benefit of U.S. Provisional Application No. 61/527,630, filed Aug. 26, 2011, which is incorporated herein by reference in its entirety.

This invention was made with Government support under contract number DE-AC02-98CH10886, awarded by the U.S. Department of Energy. The Government has certain rights in the invention.

### BACKGROUND

Alkanes are the major constituents of gasoline, diesel and jet fuels. They are also naturally produced directly from fatty acid metabolites by diverse species such as insects (as pheromones) and plants (as cuticular waxes), for example. The quantities made naturally, however, are not commercially viable. Engineered biosynthesis of alkanes may provide a renewable source of hydrocarbon biofuel. Still, the genetics and biochemistry behind the biology have remained elusive and are only now being deduced by researchers. Alkanes are made by the conversion of aldehydes in a process aided by aldehyde decarbonylase (AD or ADC). Schirmer, et al., Science (2010) 329:559-662. Even so, the use of aldehyde decarbonylase for the synthesis of alkanes even on a research scale has been problematic since the process seems to be inhibited by unknown biochemical mechanisms. Thus, what is needed are compositions and methods that provide for better production of biosynthetic alkanes.

### SUMMARY

The present compositions and methods are related to enhancing the production of alkanes and alkenes in organisms. The present compositions and methods are related to the surprising and unexpected finding that aldehyde decarbonylase is inhibited by hydrogen peroxide (H₂O₂ or “peroxide”) and that hydrogen peroxide metabolizing enzymes such as catalase can relieve the inhibition. In the absence of catalase ADC turns over approximately three times after which it becomes inactive. Warui, et al., J Am Chem Soc., (2011) 133:3319-3319. Hydrogen peroxide is converted to water and oxygen in the presence of catalase (an enzyme), effectively removing hydrogen peroxide and relieving its inhibitory effect on ADC. The present work illuminates this hitherto unknown discovery by showing that adding catalase to a reaction mixture and observing the reaction to proceed for greater than 150 turnovers in a fashion linear with time of incubation in the presence of excess aldehyde substrate and reductant NADPH, and an electron transport chain of ferredoxin NADPH reductase and ferredoxin. After observing the relief of inhibition by hydrogen peroxide in the presence of catalase we engineered a transcriptional fusion protein between catalase and ADC to create a novel hybrid polypeptide (also referred to by those of ordinary skill in the art as a chimeric protein, a fusion protein, a chimera/chimeric or a protein/fusion protein) with two domains, a catalase domain and an ADC domain (cat-ADC). This fusion protein expression construct was expressed under the control of the T-7 expression system in E. coli, in a configuration that added a His-purification tag to the cat-ADC at the C-terminus. The resulting chimeric protein was purified with the use of Ni-NTA chromatography and the highly enriched recombinant hybrid enzyme product was tested for activity. When assayed for aldehyde decarbonylase activity, the purified fusion protein was not subject to the inhibition previously seen for the native enzyme. Further, the enzyme was insensitive to added hydrogen peroxide. It is noted here that catalase not only protects the ADC from hydrogen peroxide inhibition; it also generates oxygen, a co-substrate for the ADC, thereby converting an inhibitor into a substrate.

In this regard, in one embodiment, the present method is for enhancing the production of alkanes, alkenes or other hydrocarbons in a bioengineered microorganism, said method comprising: i) transforming a microorganism to express an aldehyde decarbonylase enzyme and a catalase enzyme and, ii) culturing said transformed microorganism under conditions and for a length of time suitable for the production of alkanes. Other hydrocarbons produced by the method of the present invention may include any hydrocarbons produced after reaction of aldehyde decarbonylase with a substrate. Suitable substrates for aldehyde decarbonylase are known by those of ordinary skill in the art. Alkanes produced by the method of the present invention include, but are not limited to alkanes that comprise from 7 to 17 carbon atoms. Further, the alkanes (or other hydrocarbons) produced by the method of the present invention may be isolated from said microorganism. In a preferred embodiment, the aldehyde decarbonylase enzyme and catalase enzyme form a hybrid protein. In another embodiment the microorganism is a prokaryote or a eukaryote. When a prokaryote, preferred microorganisms are selected from cyanobacteria and E. coli.

Another embodiment of the present composition comprises an engineered hybrid protein, wherein the protein comprises an aldehyde decarbonylase enzyme domain and a hydrogen peroxide-metabolizing (catalase) enzyme domain. In another embodiment the hybrid protein consists of or consists essentially of an aldehyde decarbonylase enzyme and a catalase enzyme. In yet another embodiment, the present composition comprises an engineered microorganism comprising the hybrid protein.

Another embodiment of the present composition comprises an expression construct encoding an aldehyde decarbonylase enzyme and a catalase enzyme. In another embodiment, the present composition comprises an engineered microorganism comprising the expression construct. Further, the expression construct may encode a hybrid protein, wherein the hybrid protein comprises an aldehyde decarbonylase enzyme and a catalase enzyme.

Another embodiment of the present composition comprises a microorganism engineered to express an aldehyde decarbonylase enzyme and a catalase enzyme. In one embodiment, the aldehyde decarbonylase and catalase are expressed as a hybrid protein. In another embodiment of the present composition the microorganism is a prokaryote or a eukaryote. When a prokaryote, preferred microorganisms are selected from cyanobacteria and E. coli.

In another embodiment, the present cell-free production system relates to producing alkanes or other hydrocarbons, wherein the cell-free system comprises an aldehyde decarbonylase enzyme, a catalase enzyme and an aldehyde substrate. In another embodiment, the production system additionally comprises reductant NADPH and an electron transport chain comprising ferredoxin NADPH reductase and ferredoxin. Additionally, a chemical electron transport chain that uses PMS (phenazine methosulfate) may also be used. See, FIG. 10. In a preferred embodiment the aldehyde decarbonylase enzyme and said catalase enzyme form a hybrid protein.

In another embodiment, the present method relates to relieving hydrogen peroxide inhibition of aldehyde decarbonylase, wherein the method comprises providing a catalase enzyme in a reaction mixture wherein said reaction mixture comprises an aldehyde decarbonylase enzyme and an aldehyde substrate. In another embodiment, the said reaction mixture also comprises reductant NADPH and an electron transport chain comprising ferredoxin NADPH reductase and ferredoxin.

Other embodiments not explicitly enumerated will be evident based on the teachings herein and are considered part of the present invention.

### BRIEF DESCRIPTION OF THE FIGURES

FIG. 1 shows: Left Panel (A)—A single reaction master mix was prepared (without catalase) and allowed to react for 15 min, at which point a sample was taken and the remainder was split into 6 individual tubes. Either nothing (control), or a second dose of NADPH (NADPH), ferredoxin reductase (FNR), ferredoxin (Fd), NADPH+ferredoxin reductase+ferredoxin (electron transport chain, ETC), or ADC (ADC) was added and the reactions went for 15 min more before being terminated. Right Panel (B)—A single reaction master mix was prepared (without catalase) and allowed to react for 15 min, at which point a sample was taken and the remainder was split into 3 individual tubes. Either nothing, catalase (CAT), or more ADC (ADC) was added and the reaction went for 15 min more.

FIG. 2 shows: Decarbonylase reactions were set up and hydrogen peroxide was added for 1 min prior to the addition of enzyme. Reactions were performed in ambient air (atm) or in 100% oxygen atmosphere (100% O₂).

FIG. 3 shows: A reaction master mix was prepared without ADC. Master mix was divided into three, four-reaction aliquots and hydrogen peroxide was added to 0, 1, or 10 mM. After 1 and 10 min in the presence of H₂O₂ single reaction aliquots were removed and either buffer or catalase was added. After 1 min, reactions were initiated with ADC.

FIG. 4 shows: Construction of the CAT-ADC fusion protein and subsequent purification.

FIG. 5 shows: Catalase assays were conducted as described above.

FIG. 6 shows: A single reaction master mix was prepared for each panel.

The master mix was split three ways and either nothing (ADC or CA), catalase (ADC+Cat or CA+Cat) or 1 mM H₂O₂ (ADC+H2O2 or CA+H2O2) was added and the reactions were sampled at indicated times.

FIG. 7 shows broad substrate specificity of aldehyde decarbonylase.

FIG. 8 shows a tabular summary of the kinetic data of aldehyde decarbonylase activity with different substrates.

FIG. 9 shows the CAT-ADC fusion peptide is capable of generating a reaction for greater than 150 turnovers and that ADC and catalase is capable of generating a reaction for greater than 240 turnovers.

FIG. 10 shows that hydrogen peroxide is generated by all electron transport systems studied and catalase can relieve the inhibition.

### DETAILED DESCRIPTION

### Definitions

The present specification uses definitions of terms known by those of skill in the art (see, US Patent Publication No. 2010/0221798, for example, the definition section of which is incorporated herein). Specific definitions as known by those of skill in the art are given below for convenience. Throughout the specification, a reference may be made using an abbreviated gene name or polypeptide name, but it is understood that such an abbreviated gene or polypeptide name represents the genus of genes or polypeptides. Such gene names include all genes encoding the same polypeptide and homologous polypeptides having the same physiological function. Polypeptide names include all polypeptides that have the same activity (e.g., that catalyze the same fundamental chemical reaction).

Any accession numbers referenced herein are derived from the National Center for Biotechnology Information (NCBI) database maintained by the National Institutes of Health, U.S.A.

As used herein, the term “turnover” or “turnover number” is the number of moles of substrate that a mole of catalyst can convert before becoming inactivated. In enzyme kinetics, the same term is used to refer to the moles of substrate converted by a mole of enzyme per unit time e.g. second or minute.

As used herein, “aldehyde decarbonylase” is defined as an enzyme that catalyses the decarboxylation (more accurately, deformylation) of aldehydes to form alkanes and CO or formate (HCO₂⁻). Warui, et al., J. Am. Chem. Soc. (2011) 133:3316-3319. Aldehyde decarbonylases are also known by those of skill in the art to catalyze other substrates to produce, for example, alkenes.

As used herein, the term “biodiesel” means a biofuel that can be a substitute for diesel derived from petroleum. Biodiesel can be used in internal combustion diesel engines in either a pure form, which is referred to as “neat” biodiesel, or as a mixture in any concentration with petroleum-based diesel. Biodiesel can include esters or hydrocarbons, such as alkanes and alkenes.

As used herein, the term “biomass” refers to a carbon source derived from biological material. Biomass can be converted into a biofuel. One exemplary source of biomass is plant matter. For example, corn, sugar cane, or switchgrass can be used as biomass. Another non-limiting example of biomass is animal matter, for example, manure. Biomass also includes waste products from industry, agriculture, forestry, and households. Examples of such waste products that can be used as biomass are fermentation waste, straw, lumber, sewage, garbage, and food leftovers. Biomass also includes sources of carbon, such as carbohydrates (e.g., monosaccharides, disaccharides, or polysaccharides) and lipids.

A nucleotide sequence is “complementary” to another nucleotide sequence if each of the bases of the two sequences match and are capable of forming Watson Crick base pairs. The term “complementary strand” is used herein interchangeably with the term “complement.” The complement of a nucleic acid strand can be the complement of a coding strand or the complement of a non-coding strand. Complementation need not be complete or 100% and may be at least 50%, 60%, 70%, 80%, 90%, 95% or 99% so long as the two strands bind each other under physiological conditions.

As used herein, the term “fatty alcohol forming peptides” means a peptide capable of catalyzing the conversion of acyl-CoA to fatty alcohol, including fatty alcohol forming acyl-CoA reductase (FAR, EC 1.1.1.*), acyl-ACP reductase, acyl-CoA reductase (EC 1.2.1.50), or alcohol dehydrogenase (EC 1.1.1.1). Additionally, one of ordinary skill in the art will appreciate that some fatty alcohol forming peptides will catalyze other reactions as well. For example, some acyl-CoA reductase peptides will accept other substrates in addition to fatty acids. Such non-specific peptides are, therefore, also included. Nucleic acid sequences encoding fatty alcohol forming peptides are known in the art, and such peptides are publicly available.

Calculations of “homology” between two sequences can be performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence that is aligned for comparison purposes is at least about 30%, preferably at least about 40%, more preferably at least about 50%, even more preferably at least about 60%, and even more preferably at least about 70%, at least about 80%, at least about 90%, or about 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein, amino acid or nucleic acid “identity” is equivalent to amino acid or nucleic acid “homology”). The percent identity between the two sequences is a function of the number of identical positions shared by the sequences, taking into account the number of gaps and the length of each gap, which need to be introduced for optimal alignment of the two sequences. The comparison of sequences and determination of percent homology between two sequences can be accomplished using a mathematical algorithm, as is known by those of skill in the art.

As used herein, the term “microorganism” means prokaryotic and eukaryotic microbial species from the domains Archaea, Bacteria and Eucarya, the latter including yeast and filamentous fungi, protozoa, algae, or higher Protista. The term “microbial cell,” as used herein, means a cell from a microorganism including, but not limited to, single cell microorganisms.

As used herein, the term “purify,” “purified” or “purification” means the removal or isolation of a molecule from its environment by, for example, isolation or separation. “Substantially purified” molecules are at least about 60% free, preferably at least about 75% free and, more preferably, at least about 90% free from other components with which they are associated. As used herein, these terms also refer to the removal of contaminants from a sample. For example, the removal of contaminants can result in an increase in the percentage of aldehydes or alkanes in a sample. For example, when aldehydes or alkanes are produced in a host cell, the aldehydes or alkanes can be purified by the removal of host cell proteins. After purification, the percentage of aldehydes or alkanes in the sample is increased. The terms “purify,” “purified” and “purification” do not require absolute purity. They are relative terms. Thus, for example, when aldehydes or alkanes are produced in host cells, a purified aldehyde or purified alkane is one that is substantially separated from other cellular components (e.g., nucleic acids, polypeptides, lipids, carbohydrates, or other hydrocarbons). In another example, a purified aldehyde or purified alkane preparation is one in which the aldehyde or alkane is substantially free from contaminants, such as those that might be present following fermentation. In some embodiments, an aldehyde or an alkane is purified when at least about 50% by weight of a sample is composed of the aldehyde or alkane. In other embodiments, an aldehyde or an alkane is purified when at least about 60%, 70%, 80%, 85%, 90%, 92%, 95%, 98%, or 99% or more by weight of a sample is composed of the aldehyde or alkane.

As used herein, “transformation” and “transforming” refer to a process in which a cell's genotype is changed as a result of the cellular uptake of exogenous nucleic acid. This may result in the transformed cell expressing a recombinant form of an RNA or polypeptide. In the case of antisense expression from the transferred gene, the expression of a naturally-occurring form of the polypeptide may be disrupted.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict between references incorporated herein by reference and the present application, the present application, including definitions, will control. In addition, the materials, methods, and examples are illustrative only and not intended to be limiting.

### Description

The invention provides compositions and methods of enhancing production of hydrocarbons (such as alkanes, alkenes, and alkynes) from substrates, for example, an acyl-ACP, a fatty acid, an acyl-CoA, a fatty aldehyde or a fatty alcohol substrate (e.g., as described in US Patent Publication No. 2010/0251601 to Hu, incorporated by reference herein) provided such hydrocarbons are produced by and substrates are utilized by aldehyde decarbonylase. Such products are useful as biofuels (e.g., substitutes for gasoline, diesel, jet fuel, etc.), specialty chemicals (e.g., lubricants, fuel additive, etc.), or feedstock for further chemical conversion (e.g., fuels, polymers, plastics, textiles, solvents, adhesives, etc.). The invention is based, in part, on the identification of hydrogen peroxide as an inhibitor of aldehyde decarbonylase. Further still, the present invention contemplates the use of aldehyde decarbonylase and catalase (which enzymatically breaks down hydrogen peroxide to water and oxygen) to enhance the production of, for example, alkanes.

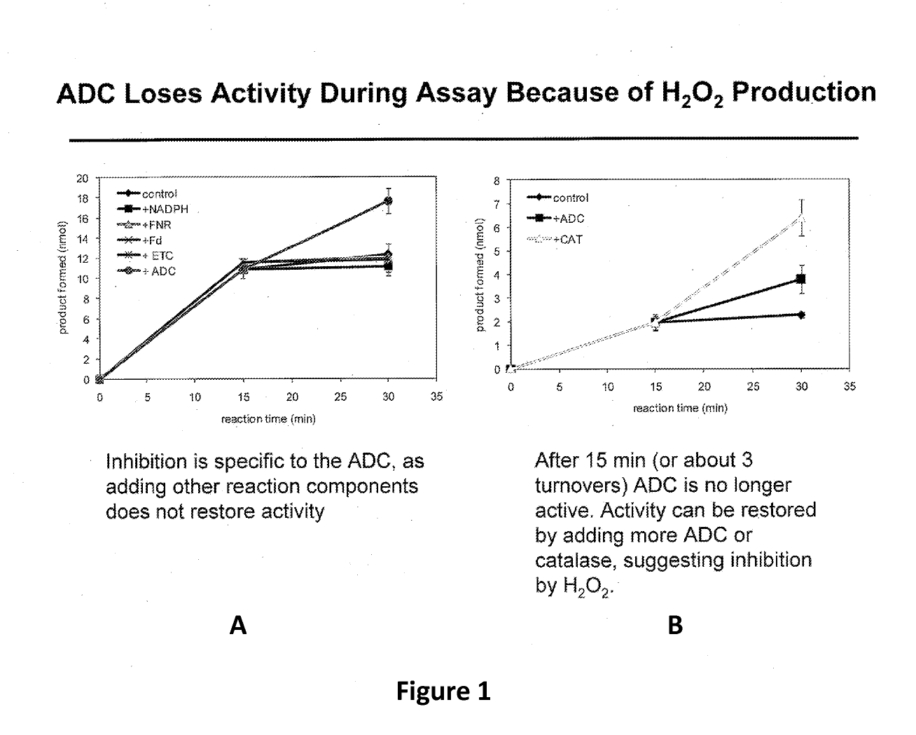

This invention provides compositions and methods for the enhanced production of alkanes by microorganisms. The present invention is related to the surprising and unexpected finding that aldehyde decarbonylase is inhibited by hydrogen peroxide (H₂O₂) (FIG. 1). In the absence of catalase ADC turns over approximately three times at which time it became inactive. Warui, et al., J Am Chem Soc., (2011) 133:3319-3319. Hydrogen peroxide can be converted to water and oxygen in the presence of catalase (an enzyme) effectively removing hydrogen peroxide and relieving its inhibitory effect on ADC. The present invention proves this hitherto unknown discovery by adding catalase to a reaction mixture and observing the reaction to proceed for greater than 150 turnovers in a fashion linear with time of incubation in the presence of excess aldehyde substrate and reductant NADPH, and an electron transport chain of ferredoxin NADPH reductase and ferredoxin. The enhanced production of alkanes (and other hydrocarbons) may be performed in microorganisms or in cell-free production systems. The present invention also provides for compositions such as hybrid aldehyde decarbonylase/catalase proteins (polypeptides), constructs encoding such hybrid fusion proteins, microorganisms engineered to contain aldehyde decarbonylase enzyme and catalase enzyme and/or expression contrasts encoding the same, wherein the aldehyde decarbonylase and catalase may or may not be in the form a hybrid fusion protein.

After observing the relief of inhibition by hydrogen peroxide in the presence of catalase, a transcriptional fusion protein between catalase and ADC was engineered to create a novel hybrid polypeptide (peptide) with two domains: a catalase domain and an ADC domain (CAT-ADC). This fusion protein expression construct was expressed under the control of the T-7 expression system in E. coli, in a configuration that added a His-purification tag to the cat-ADC at the C-terminus. The resulting chimeric protein was purified with the use of NiNTA chromatography and the highly enriched recombinant hybrid enzyme product was tested for activity. The purified fusion protein was assayed for aldehyde decarbonylase activity and the inhibition previously seen for the native enzyme was overcome. Further, the hybrid enzyme was insensitive to added hydrogen peroxide. It is noted here that catalase not only protects the ADC from hydrogen peroxide inhibition; it also generates oxygen, a co-substrate for the ADC, thereby converting an inhibitor (hydrogen peroxide) into a substrate.

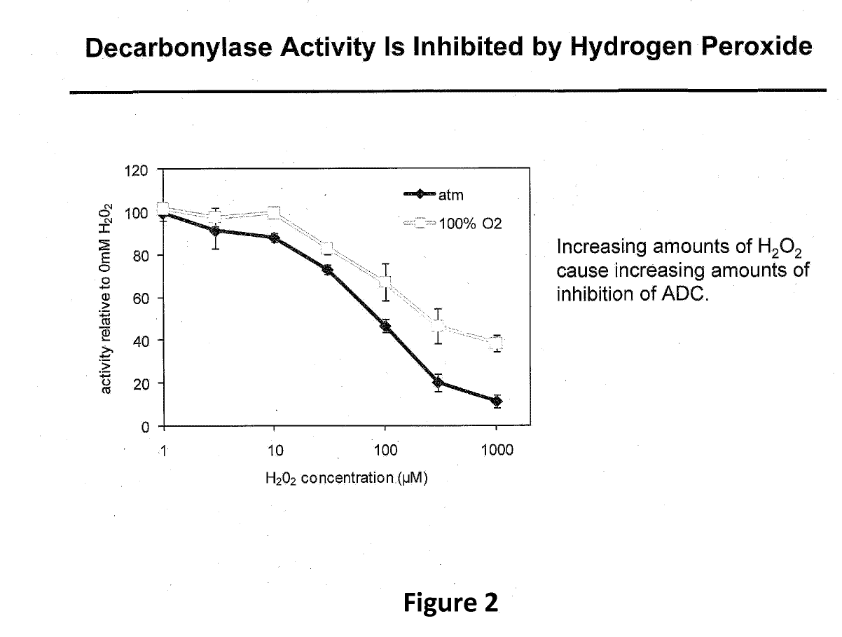

Although the present invention is not limited by theory, it is believed by the present Inventors that H₂O₂ is a competitive or non-competitive inhibitor of an aldehyde decarbonylase enzyme. FIG. 2 supports this interpretation. FIG. 2 shows lessened inhibition when reactions are in an O₂ atmosphere wherein oxygen may be out compete hydrogen peroxide. Alternative theories include the inhibition by H₂O₂ at other points in the fatty acid pathway or competition with other substrate or cofactor molecules.

H₂O₂ is a byproduct of uncoupled electron transport in photosynthetic organisms. Excess light, for instance, causes excessive reduction within the chloroplast and mechanisms inside the cell result in the generation of reactive oxygen species, H₂O₂ being one of them. Blot, et al. (Plant Physiology Review (2011) 156:1934-1954; ePub Jun. 13, 2001) describes the generation of H₂O₂ by cyanobacteria (a natural source of ADC) under high light conditions. This is very relevant because increasing light intensity generally increases the productivity of photosynthetic organisms, but one drawback with regard to alkane production is the production of H₂O₂ which could inhibit ADC. In vivo this is likely a natural feedback inhibition mechanism. In vitro, H₂O₂ is likely produced when electrons from FNR or Fd are given to oxygen instead of ADC (i.e. uncoupled reduction). In either instance the presence of hydrogen peroxide is detrimental to prolonged alkane synthesis.

The present invention is suitable for the production of alkanes and other hydrocarbons. Preferred alkanes to be produced are from 7-17 carbons in length since aldehyde decarbonylase works effectively on producing this size range of alkanes. However, the alkanes produced may be shorter or longer albeit at a lower rate of production. One of ordinary skill in the art will be able to determine production rates for the production of different sized alkanes.

Aldehyde decarbonylase enzymes have been identified from many cyanobacteria and are known in the art. For example, see Schirmer, et al., Science (2010) 329:559-562, Table 1 and supplemental materials available online, which is incorporated herein by reference. Any of the known aldehyde decarbonylase enzymes are capable of being used in the present invention. US Patent Publication No. 2010/00221798 to Schirmer, et al., provides a listing of aldehyde decarbonylase enzymes suitable for use in the present invention (see, for example, Table 1) and is incorporated herein by reference

Catalase enzymes are quite diverse and are well known to one of skill in the art. See, for example, Klotz and Loewen, Mol Biol and Evolution (2003) 20(7):1098-1112 (incorporated herein by reference), which provides a listing of most known catalase enzymes and their respective classifications. Any of the known catalase enzymes may be used in the present invention since they all catalyze the same reaction, the break down of hydrogen peroxide into water and oxygen.

This invention may be used with any microorganisms which have high rates of fatty acid synthesis or are that are suspected of being useful for the production of biofuels. The present invention can be used with bacteria such as E. coli, yeasts such as Saccoromyces cerevisiae, unicellular green algae such as Chlamydomona reinhardtii or Nannochloropsis and cynobacteria such as Synechocystis. Further, the use of this invention is not limited to microorganisms and could be applied to use in plants. Particularly oil seed crops such as canola, camalina, and soy are good examples of plants suitable for use with the compositions and methods of the present invention. Aquatic plants such as duckweed (subfamily Lemnoideae, for example) may also be useful as biofuel crops and could be used in this invention.

This invention is not limited to the production of alkanes. Any fatty acid that can be used as a substrate by aldehyde decarbonylase, and hydrocarbons made therefrom, are included in this invention in so much as the relief of inhibition of aldehyde decarbonylase by hydrogen peroxide by the methods and compositions of the present invention that leads to enhanced production of said hydrocarbons, is within the scope of the present invention. One of skill in the art, with the guidance provided by this specification, will be able to use the present invention without undue experimentation in this regard. Further details are provided below.

### Substrates

The compositions and methods described herein can be used to produce, for example, alkanes and/or alkenes from an appropriate substrate. While not wishing to be bound by a particular theory, it is believed that the methods and compositions described herein produce alkanes or alkenes from substrates via a decarbonylation mechanism. In some instances, the substrate is a fatty acid derivative, e.g., a fatty aldehyde and an alkane having particular branching patterns and carbon chain length can be produced from a fatty acid derivative, e.g., a fatty aldehyde, having those particular characteristics. In other instances, the substrate is an unsaturated fatty acid derivative, e.g., an unsaturated fatty aldehyde, and an alkene having particular branching patterns and carbon chain length can be produced from an unsaturated fatty acid derivative, e.g., an unsaturated fatty aldehyde, having those particular characteristics. Other substrates that can be used to produce alkanes and alkenes in the methods described herein are acyl-ACP, acyl-CoA, a fatty aldehyde, or a fatty alcohol, which are described in, for example, US 2010/0251601 to Hu.

### Genetic Engineering of Host Cells

One of ordinary skill in the art will realize that various host cells can be used to produce in the present invention. US Patent Publication No. 2010/0221798 lists host cells (copied below for convenience) known to those of ordinary skill in the art that are suitable for use in the present invention. A host cell can be any prokaryotic or eukaryotic cell. For example, the compositions and methods of the present invention described herein can be expressed in bacterial cells (such as E. coli), insect cells, yeast or mammalian cells (such as Chinese hamster ovary cells (CHO) cells, COS cells, VERO cells, BHK cells, HeLa cells, Cv1 cells, MDCK cells, 293 cells, 3T3 cells, or PC12 cells). Other exemplary host cells include cells from the members of the genus Escherichia, Bacillus, Lactobacillus, Rhodococcus, Pseudomonas, Aspergillus, Trichoderma, Neurospora, Fusarium, Humicola, Rhizomucor, Kluyveromyces, Pichia, Mucor, Myceliophtora, Penicillium, Phanerochaete, Pleurotus, Trametes, Chrysosporium, Saccharomyces, Schizosaccharomyces, Yarrowia, or Streptomyces. Yet other exemplary host cells can be a Bacillus lentus cell, a Bacillus brevis cell, a Bacillus stearothermophilus cell, a Bacillus licheniformis cell, a Bacillus alkalophilus cell, a Bacillus coagulans cell, a Bacillus circulans cell, a Bacillus pumilis cell, a Bacillus thuringiensis cell, a Bacillus clausii cell, a Bacillus megaterium cell, a Bacillus subtilis cell, a Bacillus amyloliquefaciens cell, a Trichoderma koningii cell, a Trichoderma viride cell, a Trichoderma reesei cell, a Trichoderma longibrachiatum cell, an Aspergillus awamori cell, an Aspergillus fumigates cell, an Aspergillus foetidus cell, an Aspergillus nidulans cell, an Aspergillus niger cell, an Aspergillus oryzae cell, a Humicola insolens cell, a Humicola lanuginose cell, a Rhizomucor miehei cell, a Mucor michei cell, a Streptomyces lividans cell, a Streptomyces murinus cell, or an Actinomycetes cell.

Other non-limiting examples of host cells are those listed in, for example, Table 1 of US Patent Application No. 2010/0221798, which is incorporated herein by reference.

In a preferred embodiment, the host cell is an E. coli cell. In a more preferred embodiment, the host cell is from E. coli strains B, C, K, or W.

Various methods are well known in the art can be used to genetically engineer host cells. The methods include the use of vectors, preferably expression vectors, containing a nucleic acid encoding a biosynthetic polypeptide described herein. As used herein, the term “vector” refers to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell and are thereby replicated along with the host genome. Moreover, certain vectors, such as expression vectors, are capable of directing the expression of genes to which they are operatively linked. In general, expression vectors used in recombinant DNA techniques are often in the form of plasmids. However, other forms of expression vectors, such as viral vectors (e.g., replication defective retroviruses, adenoviruses, and adeno-associated viruses), can also be used.

The recombinant expression vectors described herein include a nucleic acid described herein in a form suitable for expression of the nucleic acid in a host cell. The recombinant expression vectors can include one or more control sequences, selected on the basis of the host cell to be used for expression. The control sequence is operably linked to the nucleic acid sequence to be expressed. Such control sequences are described, for example, in Goeddel, Gene Expression Technology: Methods in Enzymology 185, Academic Press, San Diego, Calif. (1990). Control sequences include those that direct constitutive expression of a nucleotide sequence in many types of host cells and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors described herein can be introduced into host cells to produce polypeptides, including fusion polypeptides, encoded by the nucleic acids as described herein.

Recombinant expression vectors can be designed for expression of a biosynthetic polypeptide or variant in prokaryotic or eukaryotic cells (e.g., bacterial cells, such as E. coli, insect cells (using baculovirus expression vectors), yeast cells, or mammalian cells). Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example, by using T7 promoter regulatory sequences and T7 polymerase.

Expression of polypeptides in prokaryotes, for example, E. coli, is most often (but need not be) carried out with vectors containing constitutive or inducible promoters directing the expression of either fusion or non-fusion polypeptides. Fusion vectors add a number of amino acids to a polypeptide encoded therein, usually to the amino terminus of the recombinant polypeptide. Such fusion vectors may typically serve one, two, or three, or a combination of two or more of the following purposes: (1) to increase expression of the recombinant polypeptide; (2) to increase the solubility of the recombinant polypeptide; and (3) to aid in the purification of the recombinant polypeptide by acting as a ligand in affinity purification. In fusion expression vectors, a proteolytic cleavage site may be introduced at the junction of the fusion moiety and the recombinant polypeptide. This enables separation of the recombinant polypeptide from the fusion moiety after purification of the fusion polypeptide. Examples of such enzymes, and their cognate recognition sequences, include Factor Xa, thrombin, and enterokinase. Exemplary fusion expression vectors include pGEX (Pharmacia Biotech Inc; Smith et al., Gene (1988) 67:31-40), pMAL (New England Biolabs, Beverly, Mass.), and pRITS (Pharmacia, Piscataway, N.J.), which fuse glutathione 5-transferase (GST), maltose E binding protein, or protein A, respectively, to the target recombinant polypeptide.

Examples of inducible, non-fusion E. coli expression vectors include pTrc (Amann et al., Gene (1988) 69:301-315) and pET 11d (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HMS174(DE3) from a resident .lamda. prophage harboring a T7 gn1 gene under the transcriptional control of the lacUV 5 promoter.

One strategy to maximize recombinant polypeptide expression is to express the polypeptide in a host cell with an impaired capacity to proteolytically cleave the recombinant polypeptide (see Gottesman, Gene Expression Technology: Methods in Enzymology 185, Academic Press, San Diego, Calif. (1990) 119-128). Another strategy is to alter the nucleic acid sequence to be inserted into an expression vector so that the individual codons for each amino acid are those preferentially utilized in the host cell (Wada et al., Nucleic Acids Res. (1992) 20:2111-2118). Such alteration of nucleic acid sequences can be carried out by standard DNA synthesis techniques.

In another embodiment, the host cell is a yeast cell. In this embodiment, the expression vector is a yeast expression vector. Examples of vectors for expression in yeast S. cerevisiae include pYepSec1 (Baldari et al., EMBO J. (1987) 6:229-234), pMFa (Kurjan et al., Cell (1982) 30:933-943), pJRY88 (Schultz et al., Gene (1987) 54:113-123), pYES2 (Invitrogen Corporation, San Diego, Calif.), and picZ (Invitrogen Corp, San Diego, Calif.).

Alternatively, a polypeptide described herein can be expressed in insect cells using baculovirus expression vectors. Baculovirus vectors available for expression of proteins in cultured insect cells (e.g., Sf 9 cells) include, for example, the pAc series (Smith et al., Mol. Cell Biol. (1983) 3:2156-2165) and the pVL series (Lucklow et al., Virology (1989) 170:31-39).

In yet another embodiment, the nucleic acids described herein can be expressed in mammalian cells using a mammalian expression vector. Examples of mammalian expression vectors include pCDM8 (Seed, Nature (1987) 329:840) and pMT2PC (Kaufman et al., EMBO J. (1987) 6:187-195). When used in mammalian cells, the expression vector's control functions can be provided by viral regulatory elements. For example, commonly used promoters are derived from polyoma, Adenovirus 2, cytomegalovirus and Simian Virus 40. Other suitable expression systems for both prokaryotic and eukaryotic cells are described in chapters 16 and 17 of Sambrook et al., eds., Molecular Cloning: A Laboratory Manual. 2nd, ed., Cold Spring Harbor Laboratory, Cold Spring Harbor Laboratory Press, Cold Spring Harbor, N.Y., 1989.

Vectors can be introduced into prokaryotic or eukaryotic cells via conventional transformation or transfection techniques. As used herein, the terms “transformation” and “transfection” refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in, for example, Sambrook, et al. (supra).

For stable transformation of bacterial cells, it is known that, depending upon the expression vector and transformation technique used, only a small fraction of cells will take-up and replicate the expression vector. In order to identify and select these transformants, a gene that encodes a selectable marker (e.g., resistance to antibiotics) can be introduced into the host cells along with the gene of interest. Selectable markers include those that confer resistance to drugs, such as ampacillin, kanamycin, chloramphenicol, or tetracycline. Nucleic acids encoding a selectable marker can be introduced into a host cell on the same vector as that encoding a polypeptide described herein or can be introduced on a separate vector. Cells stably transformed with the introduced nucleic acid can be identified by drug selection (e.g., cells that have incorporated the selectable marker gene will survive, while the other cells die).

For stable transformation of mammalian cells, it is known that, depending upon the expression vector and transformation technique used, only a small fraction of cells may integrate the foreign DNA into their genome. In order to identify and select these integrants, a gene that encodes a selectable marker (e.g., resistance to antibiotics) can be introduced into the host cells along with the gene of interest. Preferred selectable markers include those which confer resistance to drugs, such as G418, hygromycin, and methotrexate. Nucleic acids encoding a selectable marker can be introduced into a host cell on the same vector as that encoding a polypeptide described herein or can be introduced on a separate vector. Cells stably transformed with the introduced nucleic acid can be identified by drug selection (e.g., cells that have incorporated the selectable marker gene will survive, while the other cells die).

In certain methods, an aldehyde biosynthetic polypeptide and an alkane or alkene biosynthetic polypeptide are co-expressed in a single host cell. In alternate methods, an aldehyde biosynthetic polypeptide and an alcohol dehydrogenase polypeptide are co-expressed in a single host cell.

### Fermentation

The production and isolation of products of the present invention can be enhanced by employing beneficial fermentation techniques. One method for maximizing production while reducing costs is increasing the percentage of the carbon source that is converted to hydrocarbon products.

During normal cellular lifecycles, carbon is used in cellular functions, such as producing lipids, saccharides, proteins, organic acids, and nucleic acids. Reducing the amount of carbon necessary for growth-related activities can increase the efficiency of carbon source conversion to product. This can be achieved by, for example, first growing host cells to a desired density (for example, a density achieved at the peak of the log phase of growth). At such a point, replication checkpoint genes can be harnessed to stop the growth of cells. Specifically, quorum sensing mechanisms (reviewed in Camilli et al., Science 311:1113, 2006; Venturi FEMS Microbio. Rev. 30:274-291, 2006; and Reading et al., FEMS Microbiol. Lett. 254:1-11, 2006) can be used to activate checkpoint genes, such as p53, p21, or other checkpoint genes.

Genes that can be activated to stop cell replication and growth in E. coli include umuDC genes. The overexpression of umuDC genes stops the progression from stationary phase to exponential growth (Murli et al., J. of Bact. 182:1127, 2000). UmuC is a DNA polymerase that can carry out translesion synthesis over non-coding lesions—the mechanistic basis of most UV and chemical mutagenesis. The umuDC gene products are involved in the process of translesion synthesis and also serve as a DNA sequence damage checkpoint. The umuDC gene products include UmuC, UmuD, umuD′, UmuD′_{2C}, UmuD′₂, and UmuD₂. Simultaneously, product-producing genes can be activated, thus minimizing the need for replication and maintenance pathways to be used while an aldehyde, alkane and/or alkene is being made. Host cells can also be engineered to express umuC and umuD from E. coli in pBAD24 under the prpBCDE promoter system through de novo synthesis of this gene with the appropriate end-product production genes.

The percentage of input carbons converted to, for example, alkanes and/or alkenes can be a cost driver. The more efficient the process is (i.e., the higher the percentage of input carbons converted to alkanes and/or alkenes), the less expensive the process will be. For oxygen-containing carbon sources (e.g., glucose and other carbohydrate based sources), the oxygen must be released in the form of carbon dioxide. For every 2 oxygen atoms released, a carbon atom is also released leading to a maximal theoretical metabolic efficiency of approximately 34% (w/w) (for fatty acid derived products). This figure, however, changes for other hydrocarbon products and carbon sources. Typical efficiencies in the literature are approximately less than 5%. Host cells engineered to produce alkanes and/or alkenes via the compositions and methods of the present invention can have greater than about 1, 3, 5, 10, 15, 20, 25, and 30% efficiency. In one example, host cells can exhibit an efficiency of about 10% to about 25%. In other examples, such host cells can exhibit an efficiency of about 25% to about 30%. In other examples, host cells can exhibit greater than 30% efficiency.

In one example, the fermentation chamber can enclose a fermentation that is undergoing a continuous reduction. In this instance, a stable reductive environment can be created. The electron balance can be maintained by the release of carbon dioxide (in gaseous form). Efforts to augment the NAD/H and NADP/H balance can also facilitate in stabilizing the electron balance. The chemical reducing system utilizing PMS and NADH may also be used. The availability of intracellular NADPH can also be enhanced by engineering the host cell to express an NADH:NADPH transhydrogenase. The expression of one or more NADH:NADPH transhydrogenases converts the NADH produced in glycolysis to NADPH, which can enhance the production of alkanes and/or alkenes.

For small scale production, the engineered host cells can be grown in batches of, for example, around 100 mL, 500 mL, 1 L, 2 L, 5 L or 10 L, fermented and induced to produce desired products. For example, E. coli BL21(DE3) cells harboring pBAD24 (with ampicillin resistance and, for example, a fusion protein of the present invention) as well as pUMVC1 (with kanamycin resistance and the acetyl CoA/malonyl CoA overexpression system) can be incubated overnight in 2 L flasks at 37° C. shaken at >200 rpm in 500 mL LB medium supplemented with 75 μg/mL ampicillin and 50 μg/mL kanamycin until cultures reach an OD₆₀₀ of >0.8. Upon achieving an OD₆₀₀ of >0.8, the cells can be supplemented with 25 mM sodium proprionate (pH 8.0) to activate the engineered gene systems for production and to stop cellular proliferation by activating UmuC and UmuD proteins. Induction can be performed for 6 hrs at 30° C. After incubation, the media can be examined for, e.g., alkanes and/or alkenes using GC-MS.

For large scale production, the engineered host cells can be grown in batches of 10 L, 100 L, 1000 L or larger; fermented; and induced to produce desired alkanes and/or alkenes based on the substrate. For example, E. coli BL21(DE3) cells harboring pBAD24 (with ampicillin resistance and for example, a fusion protein of the present invention) as well as pUMVC1 (with kanamycin resistance and the acetyl-CoA/malonyl-CoA overexpression system) can be incubated from a 500 mL seed culture for 10 L fermentations (5 L for 100 L fermentations, etc.) in LB media (glycerol free) with 50 ng/mL kanamycin and 75 ng/mL ampicillin at 37° C., and shaken at >200 rpm until cultures reach an OD₆₀₀ of >0.8 (typically 16 hrs). Media can be continuously supplemented to maintain 25 mM sodium proprionate (pH 8.0) to activate the engineered gene systems for production and to stop cellular proliferation by activating umuC and umuD proteins. Media can be continuously supplemented with glucose to maintain a concentration 25 g/100 mL.

After the first hour of induction, aliquots of no more than 10% of the total cell volume can be removed each hour and allowed to sit without agitation to allow the product (e.g., alkanes) to rise to the surface and undergo a spontaneous phase separation. The product can then be collected, and the aqueous phase returned to the reaction chamber. The reaction chamber can be operated continuously. When the OD₆₀₀ drops below 0.6, the cells can be replaced with a new batch grown from a seed culture.

### Cell-Free Methods

In some methods described herein, a product (e.g., an alkane) can be produced using an isolated or purified polypeptide described herein and a substrate described herein. For example, a host cell can be engineered to express a fusion protein, for example, as described herein. The host cell can be cultured under conditions suitable to allow expression of the polypeptide. Cell free extracts can then be generated using known methods. For example, the host cells can be lysed using detergents or by sonication. The expressed polypeptides can be purified using known methods. After obtaining the cell free extracts, substrates described herein can be added to the cell free extracts and maintained under conditions to allow conversion of the substrates to alkanes and/or alkenes. The alkanes and/or alkenes can then be separated and purified using known techniques.

### Post-Production Processing

The alkanes and/or alkenes produced during fermentation can be separated from the fermentation media. Any known technique for separating alkanes and/or alkenes from aqueous media can be used.

### Fuel Compositions and Specialty Chemical Compositions

The products described herein may be used as a fuel or converted into a fuel or may be used as a specialty chemical. One of ordinary skill in the art will appreciate that, depending upon the intended purpose of the fuel or specialty chemical, the products of the present invention (e.g., alkanes) can be produced and used.

### EXEMPLIFICATION

### Experimental

The initial experiments showed that aldehyde decarbonylase (ADC) loses activity because of the production of hydrogen peroxide (H₂O₂). Hydrogen peroxide was added to enzyme assays as described below. ADC was selectively inhibited by H₂O₂ as shown in FIG. 1A. ADC activity was inhibited after about 15 min (or about 3 turnovers, see FIG. 1B). FIG. 1B also shows the restoration of ADC activity after the addition of catalase. Addition of more ADC also restored ACD activity showing that the inhibition was not of a general nature.

The data in FIG. 2 confirms that the inhibition of ADC is by H₂O₂. Increasing amounts of H₂O₂ caused increasing inhibition of ADC. Inhibition was lessened in a 100% O₂ (1250 μM) atmosphere, and enhanced in a 4% (50 μM) atmosphere lending further support to the finding that H₂O₂ inhibits ADC. O₂ is a substrate of ADC and high levels would likely outcompete H₂O₂ for ADC binding sites

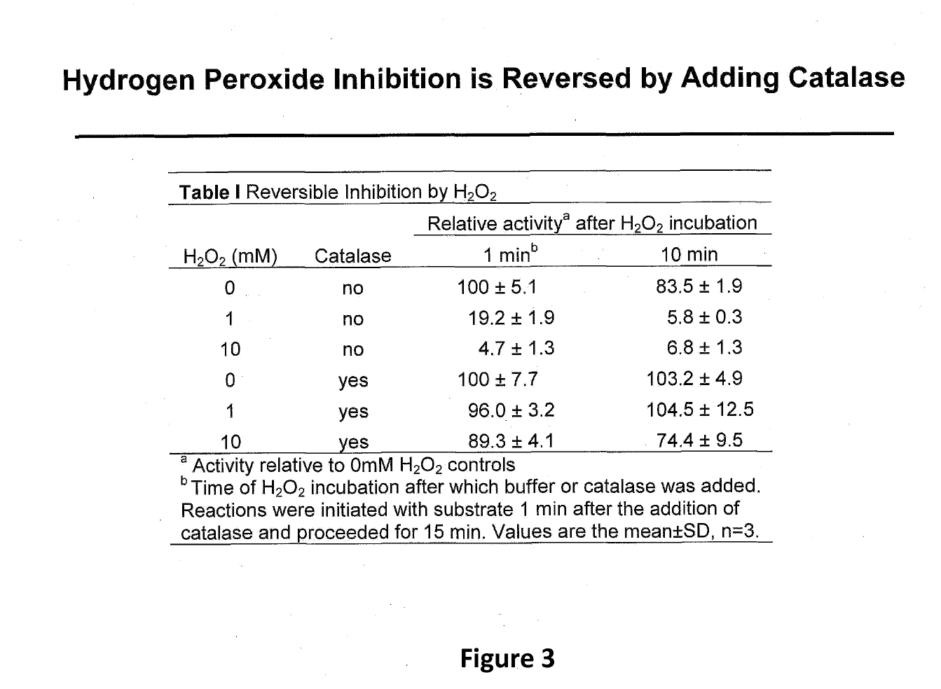

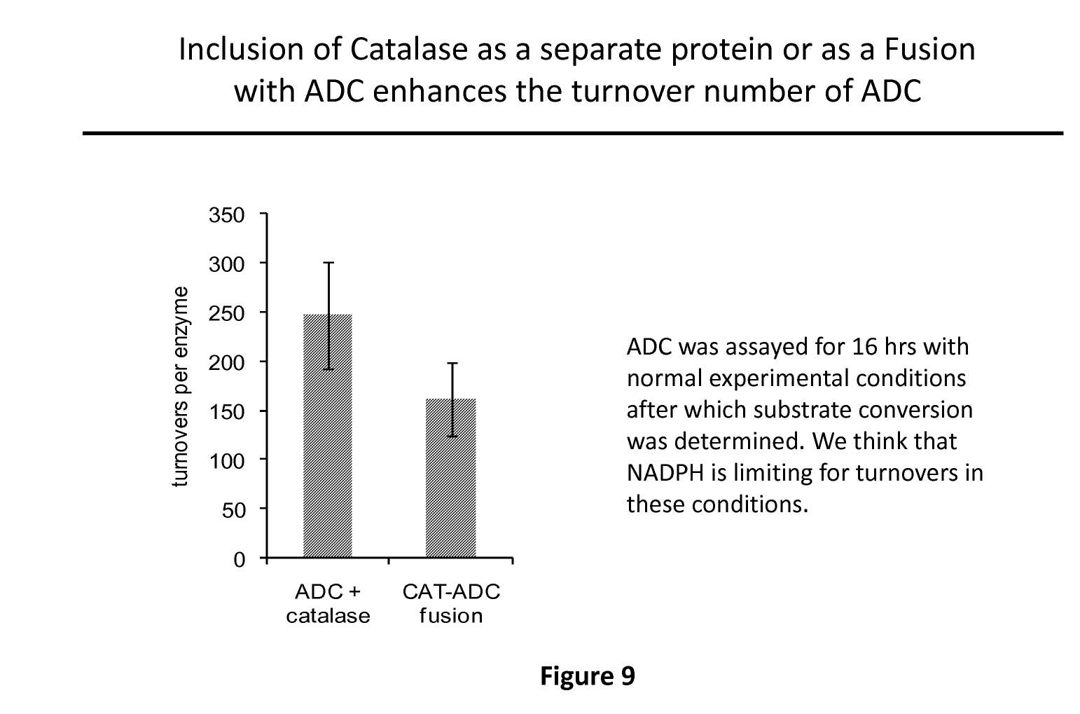

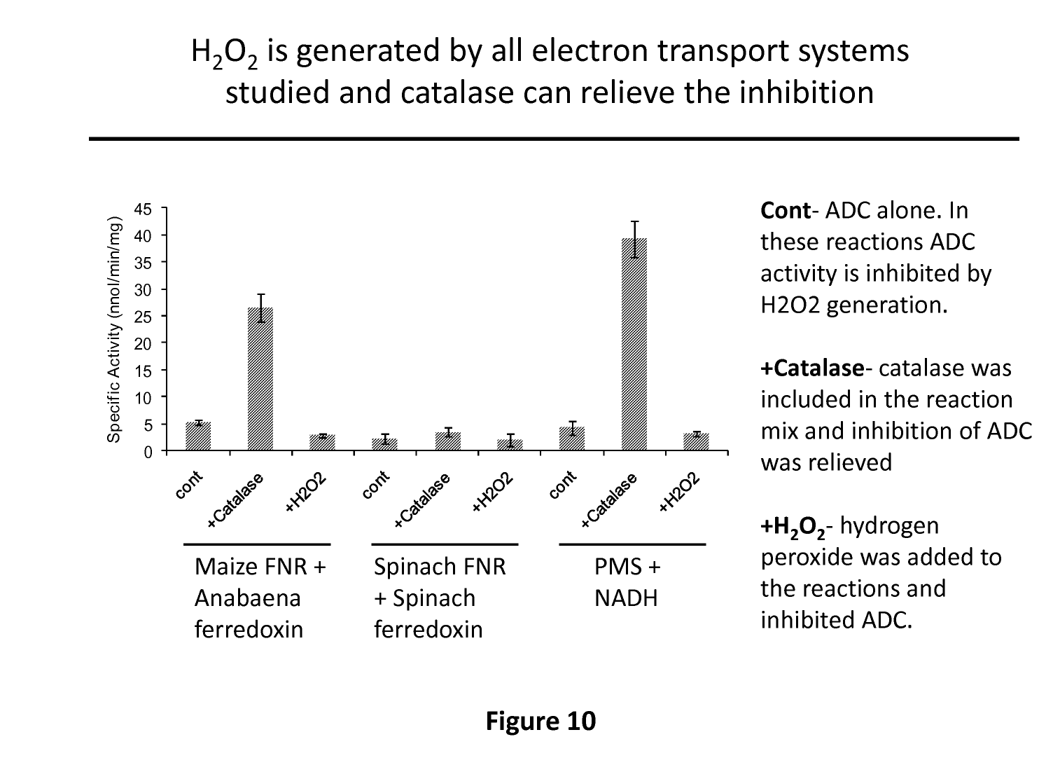

As shown in FIG. 3, inhibition of ADC by H₂O₂ is reversed by adding catalase. In this experiment catalase was added to assays wherein 0 mM, 1 mM or 10 mM of H₂O₂ was used to induce inhibition of ADC. Catalase was effective in overcoming the H₂O₂ induced inhibition of ADC. FIG. 9 shows that the reaction can proceed for greater than 150 turnovers in the presence of catalase. In FIG. 9 reactions were set up as previously described, but instead of stopping the reactions after 15 min, they were allowed to react for 16 hrs. In FIG. 10 it is shown that hydrogen peroxide is generated by all electron transport systems studied and catalase relieves the inhibition. The experiments were performed as described, except that in one case, Maize FNR and anabaena Fd were replaced with 1 U/ml Spinach FNR and 2.23 mg/mL Spinach ferredoxin, both from Sigma-Aldrich. In the other case, FNR, ferredoxin, and NADPH were omitted and instead 0.075 mM PMS and 0.75 mM NADH were included.

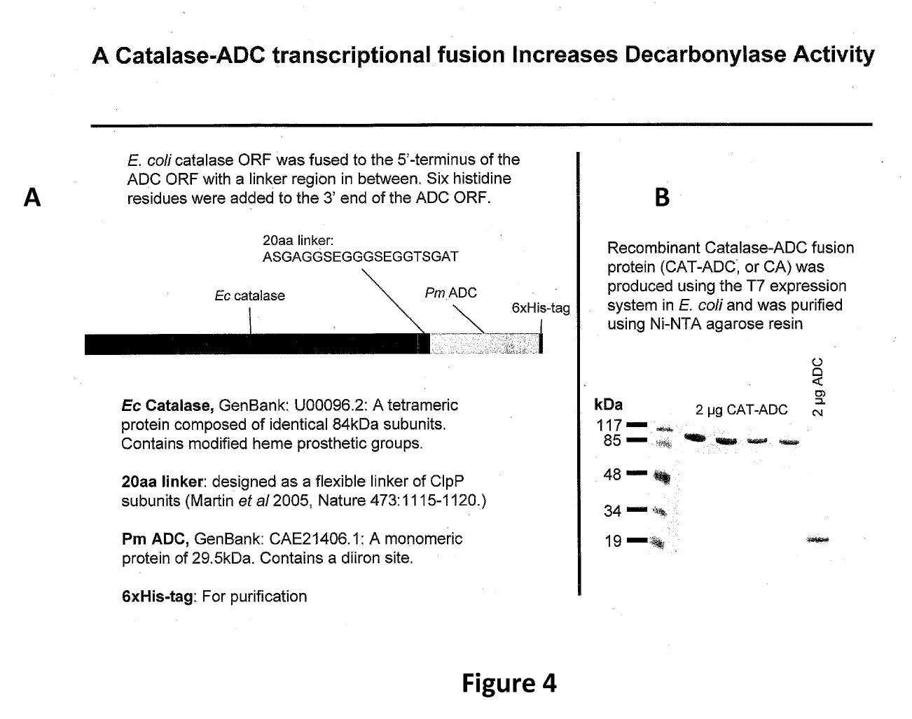

A catalase transcriptional fusion was produced as described below. FIG. 4A shows a representation of the fusion. The Catalase used was Ec catalase (GenBank Accession No. U00092.2) though the present invention is not limited to the use of this particular catalase. The ADC used was Pm ADC (GenBank Accession No. CAE21406.1). The ADC contains a diiron site. ADCs utilizing other metals are also suitable for use. For example, ribonucleotide reductase, which is normally a diiron protein can also use Mn (manganese) (Metallomics, 2011, 3(2):110-120, ePub 2001 Jan. 25). The catalase and ADC sequences were connected by a 20 amino acid linker: ASGAGGSEGGGSEGGTSGAT [SEQ ID NO: 8]. A 6×His-tag was added to the 3′ end for simplified purification. The recombinant catalase-ADC fusion (hybrid) protein (CAT-ADC or CA) was produced using the T7 expression system in E. coli and was purified using Ni-NTA agarose resin. FIG. 4B shows the successful production of the fusion protein by SDS gel electrophoresis.

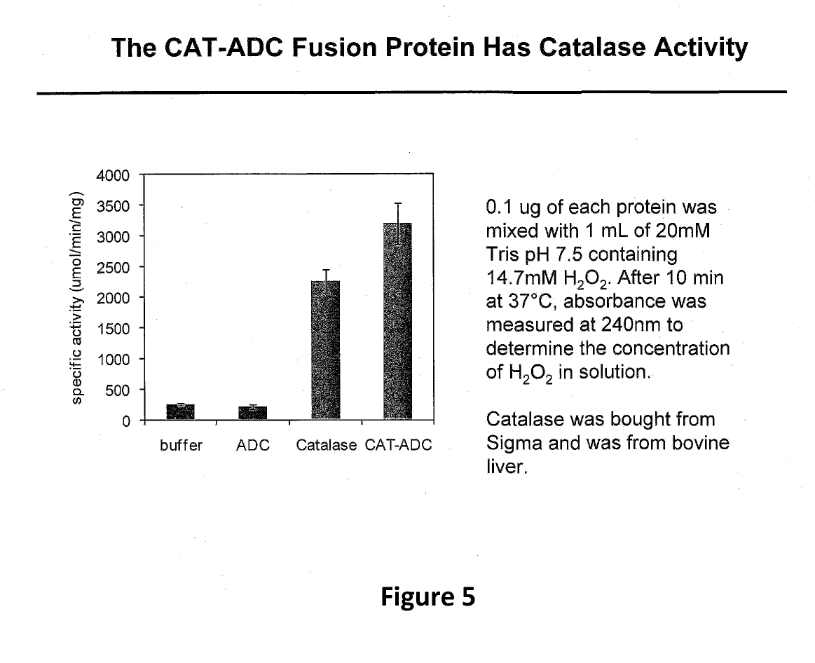

As shown in FIG. 5, the CAT-ADC fusion protein has catalase activity. 0.1 μg of each protein was mixed with 1 mL of 20 mM Tris pH 7.5 containing 14.7 mM H₂O₂. After 10 min at 37° C. absorbance was measured at 240 nm to determine the concentration of H₂O₂ in solution. Catalase for the positive control was purchased from Sigma/Aldrich (St. Louis, Mo.).

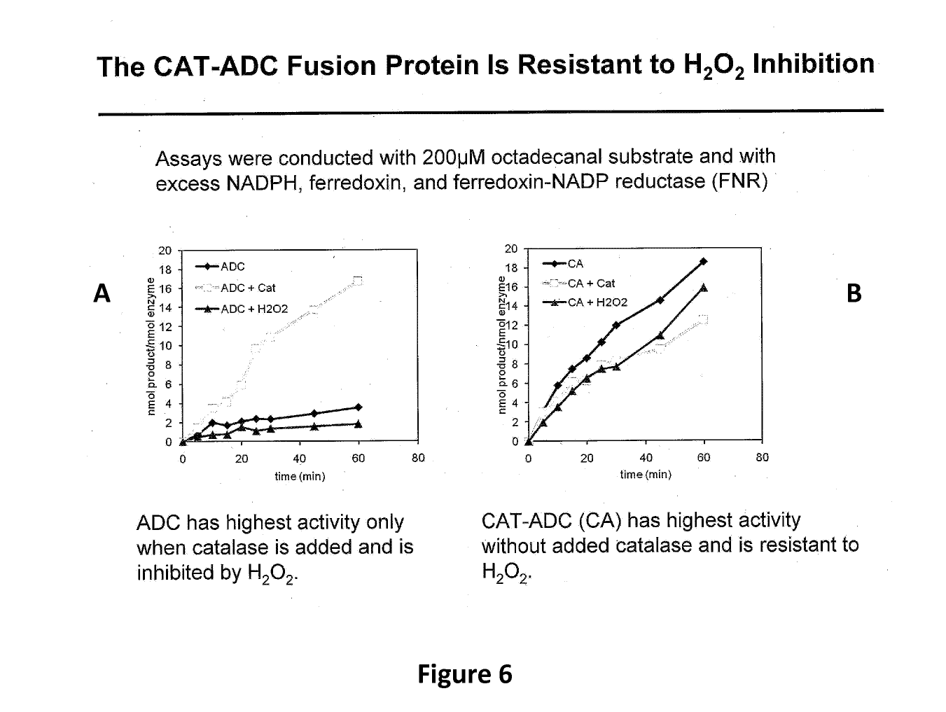

An experiment was conducted to determine if the CAT-ADC fusion protein was resistant to H₂O₂ inhibition. FIG. 6 shows assays that were conducted with 200 μM octadencanal substrate and with excess NADPH, ferredoxin and ferridoxin-NADP reductase (FNR). FIG. 6A shows that ADC had the highest activity only when catalase was added and was inhibited by H₂O₂. FIG. 6B shows that CAT-ADC(CA) had the highest activity without added catalase and was resistant to H₂O₂ inhibition.

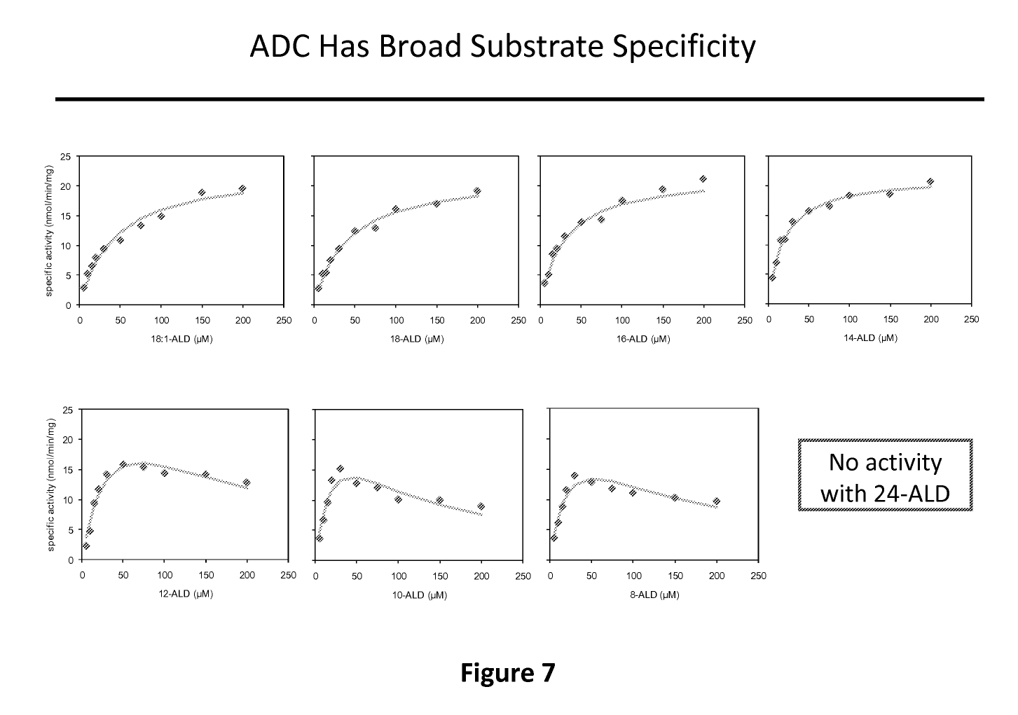

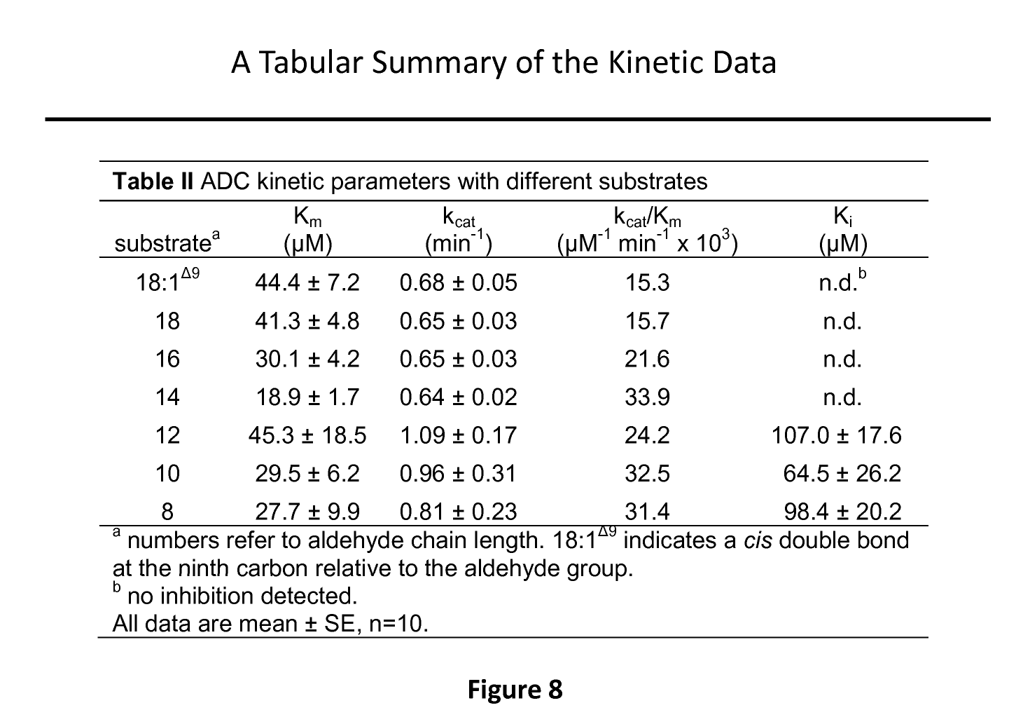

FIG. 7 shows broad substrate specificity of aldehyde decarbonylase for aldehydes of different lengths. FIG. 8 shows a tabular summary of the kinetic data of aldehyde decarbonylase activity with different substrates.

### Experimental Procedures

### Vector Construction

The pSpeedET T7 inducible bacterial expression plasmid containing the coding sequence for Prochlorococcus marinus aldehyde decarbonylase (ADC, GenBank: CAE21406.1 [SEQ ID NO: 1]) fused with an N-terminal MGSDKIHHHHHHENLYFQG [SEQ ID NO: 2] tag as constructed by the Joint Center for Structural Genomics, was obtained from the DNASU plasmid repository. The E. coli catalase (katE GenBank: U00096.2 [SEQ ID NO: 3]) ADC fusion protein (CAT-ADC) was constructed by overlap extension PCR. Primers for catalase were: forward—AATTGGCATATGTCGCAACATAACGAAAAGAACC [SEQ ID NO: 4] and reverse—ACCACCTTCAGAGCCACCGCCTTCAGAGCCGCCCGCACCAGACGCGGCAGGAAT TTTGTCAATCTTAGG [SEQ ID NO: 5]. Primers for ADC were: forward—GGCTCTGAAGGCGGTGGCTCTGAAGGTGGTACCTCTGGTGCGACCATGCCTACGC TTGAGATGCCT [SEQ ID NO: 6] and reverse—AATTGGCTCGAGTCAGTGGTGGTGGTGGTGGTGGCTCACAAGAGCTGCC [SEQ ID NO: 7]. The final construct contained catalase followed by a 20 amino acid flexible linker domain (ASGAGGSEGGGSEGGTSGAT [SEQ ID NO: 8]) (Martin et al 2005, Nature 473:1115-1120.) followed by ADC and finally a C-terminal hexahistidine tag and was cloned into the NdeI and XhoI sites of pET24b.

### Protein Expression and Purification

E. coli BL21 (DE3) Gold cells containing various plasmids were grown at 37° C. to an OD₆₀₀ of 0.4, were induced with 0.4 mM IPTG, and were grown for an additional 4 hours at 37° C. for ADC or 30° C. for CAT-ADC. Cell pellets were suspended in 20 mM Tris-Cl pH 7.5, 150 mM NaCl, 20 mM imidazole, 5 mM MgCl₂, and 0.1 mg/mL DNase and were lysed using a French pressure cell. Cellular debris was removed by centrifugation at 40,000×g for 20 min. Recombinant proteins were purified from the soluble fraction with Ni-NTA resin (Qiagen). Wash and elution buffers were 20 mM Tris-Cl pH 7.5, 300 mM NaCl, and 25 or 250 mM imidazole, respectively. Eluted proteins were immediately exchanged into 20 mM HEPES pH 7.8, 200 mM NaCl using PD-10 desalting columns. Protein concentration was determined with Bradford Assay (Sigma).

### Enzyme Assays

Typical decarbonylase assays were 0.25 mL and contained 25 mM Tris-Cl pH 7.5, 0.1% Triton X-100, 1 mM DTT, 50 μg/mL maize root ferredoxin and 1 U/mL anabaena vegetative ferredoxin reductase (Cahoon et al. 1997, PNAS 94:4872-4877.), 2 mM NADPH, 200 μM octadecanal, and between 0.2-5 μM ADC or CAT-ADC. A 20 mM octadecanal stock was freshly prepared by sonicating powder in 10% Triton X-100. Octadecanal was obtained from ISCA technologies. When indicated catalase (Sigma C-9322) was added to a final concentration of 4 mg/mL from a 20 mg/mL stock dissolved in 100 mM PIPES pH 6.0. For assays performed in 100% oxygen a reaction master mix without NADPH and a separate NADPH solution were prepared by repeated purging of the sample cell with 100% O₂ and vacuum with the use of a Schlenk line. Reactions were initialed by addition of either enzyme, substrate, or NADPH and were incubated at 37° C. and stopped by the addition of an equal volume of ethyl acetate. The organic phase was separated by GC/MS on an HP-5 ms column with oven temperature increasing from 75° C. to 320° C. at 40° C./min with a flow rate of 1.3 ml/min. Substrate and product were identified by comparison to authentic standards.

Catalase assays were 1 mL and contained 20 mM Tris-Cl pH 7.5, 14.7 mM H₂O₂. Assays were initiated by the addition of 0.1 μg of protein and were incubated at 37° C. H₂O₂ concentration was determined by measuring absorbance at 240 nm and using a molar extinction coefficient of 43.6 M⁺¹ cm⁻¹.

1. A method for enhancing the production of one or more of alkanes and alkenes in a bioengineered microorganism, said method comprising: i) transforming a microorganism to express an aldehyde decarbonylase enzyme and a peroxide metabolizing enzyme and, ii) culturing said transformed microorganism under conditions and for a length of time suitable for the production of one or more of alkanes and alkenes.

2. The method of claim 1, wherein said alkanes and alkenes comprise from 7 to 17 carbon atoms.

3. The method of claim 1, wherein the alkanes and alkenes produced by said method are isolated from said microorganism.

4. The method of claim 1, wherein aldehyde decarbonylase enzyme and said peroxide metabolizing enzyme form a hybrid protein.

5. The method of claim 1, wherein said microorganism is a prokaryote.

6. The method of claim 1, wherein said microorganism is a eukaryote.

7. The method of claim 5, wherein said microorganism is selected from the group consisting of cyanobacteria and E. coli.

8. A composition comprising an engineered hybrid protein, said protein comprising an aldehyde decarbonylase enzyme and a peroxide metabolizing enzyme.

9. An engineered microorganism comprising the hybrid protein of claim 8.

10. A composition comprising an expression construct encoding an aldehyde decarbonylase enzyme and a peroxide metabolizing enzyme.

11. An engineered microorganism comprising the expression construct of claim 10.

12. The composition of claim 10, wherein the expression construct encodes a hybrid protein.

13. A composition comprising a microorganism engineered to express an aldehyde decarbonylase enzyme and a peroxide metabolizing enzyme.

14. The composition of claim 13, wherein the aldehyde decarbonylase and peroxide metabolizing enzyme are expressed as a hybrid protein.

15. The composition of claim 13, wherein the microorganism is a prokaryote.

16. The composition of claim 13, wherein the microorganism is a eukaryote.

17. The method of claim 13, wherein said microorganism is selected from the group consisting of cyanobacteria and E. coli.

18. A cell-free production system for producing alkanes and alkenes, said cell-free system comprising an aldehyde decarbonylase enzyme, a peroxide metabolizing enzyme and an aldehyde substrate.

19. The cell-free system of claim 18, wherein said system additionally comprises reductant NADPH and an electron transport chain comprising ferredoxin NADPH reductase and ferredoxin.

20. The cell-free system of claim 18, wherein said aldehyde decarbonylase enzyme and said peroxide metabolizing enzyme form a hybrid protein.

21. A method for relieving hydrogen peroxide inhibition of aldehyde decarbonylase, said method comprising providing a peroxide metabolizing enzyme in a reaction mixture wherein said reaction mixture comprises an aldehyde decarbonylase enzyme and an aldehyde substrate.

22. The method of claim 21, wherein said reaction mixture also comprises reductant NADPH and an electron transport chain comprising ferredoxin NADPH reductase and ferredoxin.

23. The method according to claim 1 wherein the peroxide metabolizing enzyme is catalase.

24. The composition according to claim 8 wherein the peroxide metabolizing enzyme is catalase.

25. The composition according to claim 10 wherein the peroxide metabolizing enzyme is catalase.

26. The composition according to claim 13 wherein the peroxide metabolizing enzyme is catalase.

27. The composition according to claim 18 wherein the peroxide metabolizing enzyme is catalase.

28. The composition according to claim 21 wherein the peroxide metabolizing enzyme is catalase.
